(19) United States
(12) Patent Application Publication
(10) Pub. No.: US 2010/0010618 A1
(43) Pub. Date: Jan. 14, 2010

(54) Overlapping Stent

(75) Inventor: Timothy S. Girton, Edina, MN (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/169,494

(22) Filed: Jul. 8, 2008

(51) Int. Cl. A61F 2/82

(52) U.S. Cl. 623/1.16

(57) ABSTRACT

A stent defines a plurality of holes with a therapeutic agent deposited therein and has a plurality of members where at least some of the members overlap when the stent is in the unexpanded state

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patients skin, or by a “cut down” technique in which the entry blood vessel is exposed by minor surgical means.

Stents, grafts, stent-grafts, vena cava filters, expandable frameworks, and similar implantable medical devices are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists

All US patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

### BRIEF SUMMARY OF THE INVENTION

In at least one embodiment, the invention is directed to a stent defining a plurality of holes with a therapeutic agent deposited therein and comprising a plurality of members where at least some of the members overlap when the stent is in the unexpanded state.

These and other embodiments which characterize the invention are pointed out with particularity in the claims annexed hereto and forming a part hereof. However, for further understanding of the inventions its advantages and objectives obtained by its use, reference can be made to the drawings which form a further part hereof and the accompanying descriptive matter, in which there is illustrated and described an embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a flat view of a stent with overlapping member in the unexpanded state.

FIG. 2 is a flat view of the stent of FIG. 1 in the expanded state

FIG. 3 is a flat view of a stent with over lapping members in the unexpanded state.

FIG. 4 is a flat view of the stent of FIG. 3 in the expanded state.

FIG. 5 is a flat view of a stent with overlapping members in the unexpanded state.

FIG. 6 is a flat view of the stent of FIG. 5 in the expanded state.

FIG. 7 is a flat view of a stent with overlapping member in the unexpanded state.

FIG. 8 is a flat view of the stent of FIG. 7 in the expanded state.

FIG. 9 is a flat view of a stent with overlapping members in the unexpanded state.

FIG. 10 is a flat view of the stent of FIG. 9 in the expanded state.

FIG. 11 is a flat view of a stent with a first section with overlapping members and a second section with no overlapping members

FIG. 12 is the flat view of the stent of FIG. 11 in the expanded state.

### DETAILED DESCRIPTION OF THE INVENTION

While this invention may be embodied in many different forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

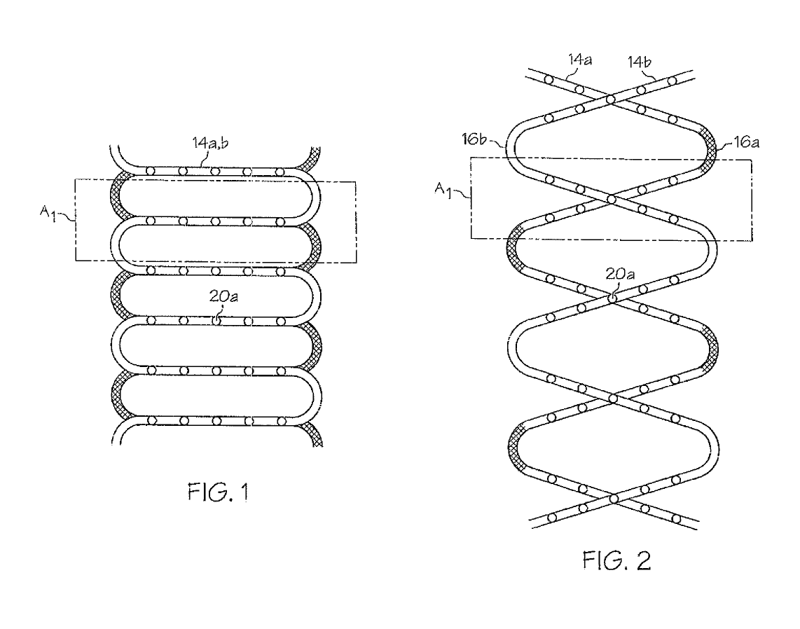

As shown in the figures, the invention is directed to a stent defining a plurality of holes 20 with a therapeutic agent deposited therein and comprising a plurality of members 12 where some of the members 12 overlap when the stent 10 is in the unexpanded state. It is within the scope of the invention for any therapeutic agent or combination thereof to be deposited into the holes 20. A non-limiting list of therapeutic agents is provided below. In some embodiments, therapeutic agent is only deposited in the holes 20. In other embodiments, therapeutic agent is deposited into the holes 20 and onto portion(s) of the surface(s) of the member 12 that are not overlapped by another member 12. Thus, for example, in FIG. 1, a therapeutic agent can be deposited onto the outer surface of turn 16a because it is not overlapped by another member 12, as indicated by cross-hatching. Note that the outer surface of turn 16b can also have therapeutic agent deposited thereon because it is not overlapped by another member 12.

FIGS. 1-12 are examples of different stent patterns where at least one portion of adjacent members 12 overlap when the stent 10 is in the unexpanded state. As used in this application, a first member 12 overlaps an adjacent member 12 when either a portion of the first member 12 overlaps, or is positioned above, the adjacent member 12, or when a portion of the first member 12 is overlapped by, or is positioned below, the adjacent member 12. Different ways members 12 can overlap are discussed in greater detail below.

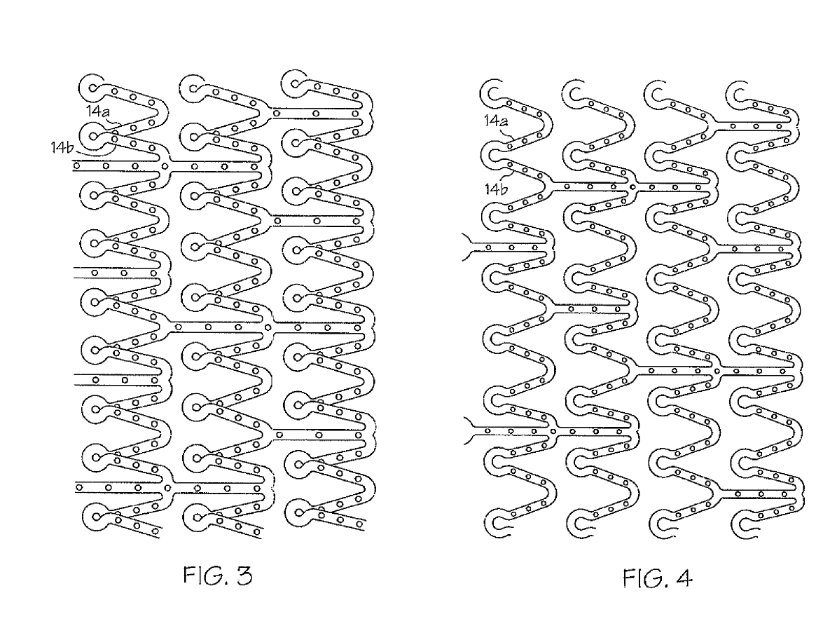

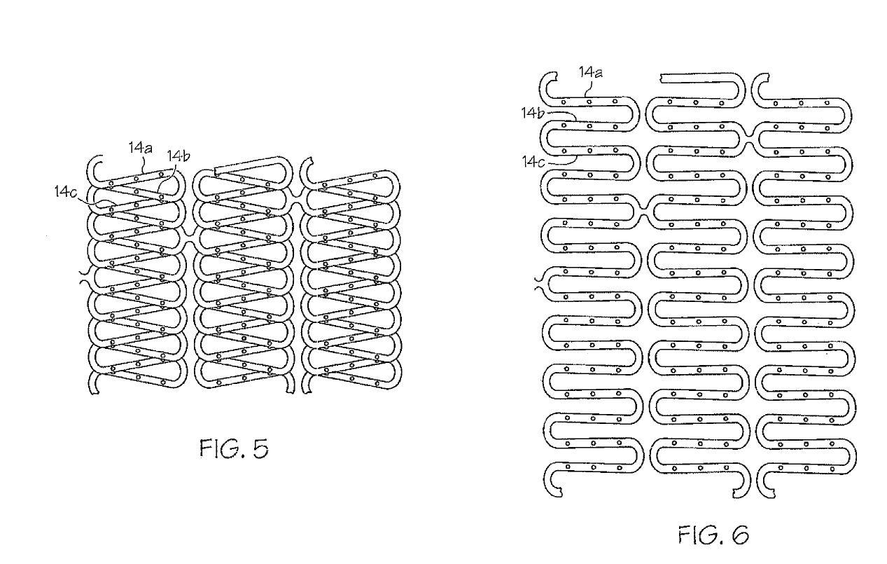

As used in this application, members 12 include struts 14, turns 16, and connectors 18. The adjacent struts 14 are engaged by turns 16. It is within the scope of the invention for struts 14, turns 16, connectors 18 and any combination thereof to have holes 20. As shown in FIG. 5, only the struts 14 have holes 20. In FIG. 3, the struts 14 and the connectors 18 have holes 20. The holes 10 have a density. As used in this application, the density of holes 20 refers to the number of holes 20 per unit of area. In some embodiments, the density of holes 20 when the stent 10 is in the unexpanded state is less than the density of holes 20 when the stent 10 is in the expanded state. For example, in the stent embodiment shown in FIGS. 1 and 2, area A1 has a density of five holes 20 when the stent 10 is in an unexpanded state and a density of ten holes 20 when the stent 10 is in the unexpanded state

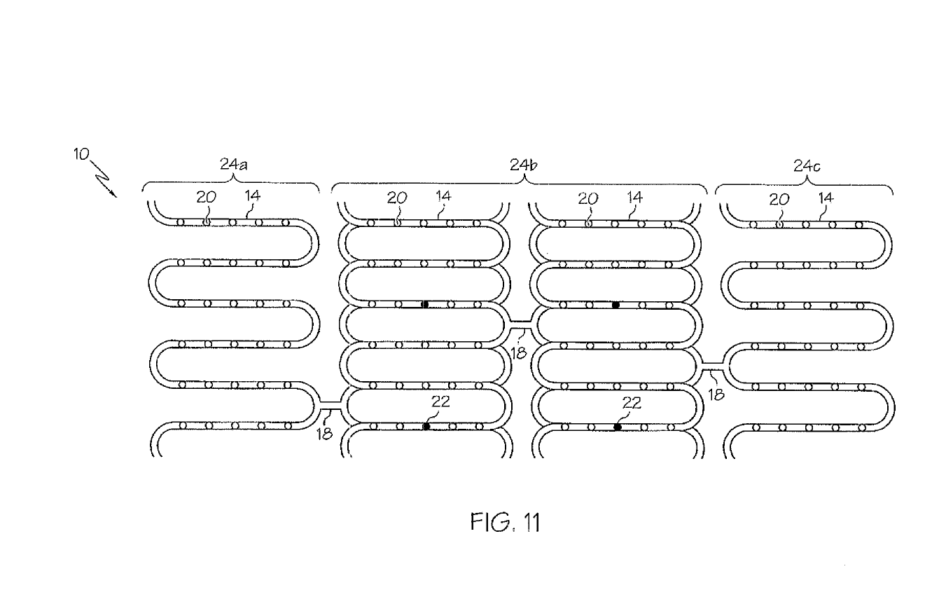

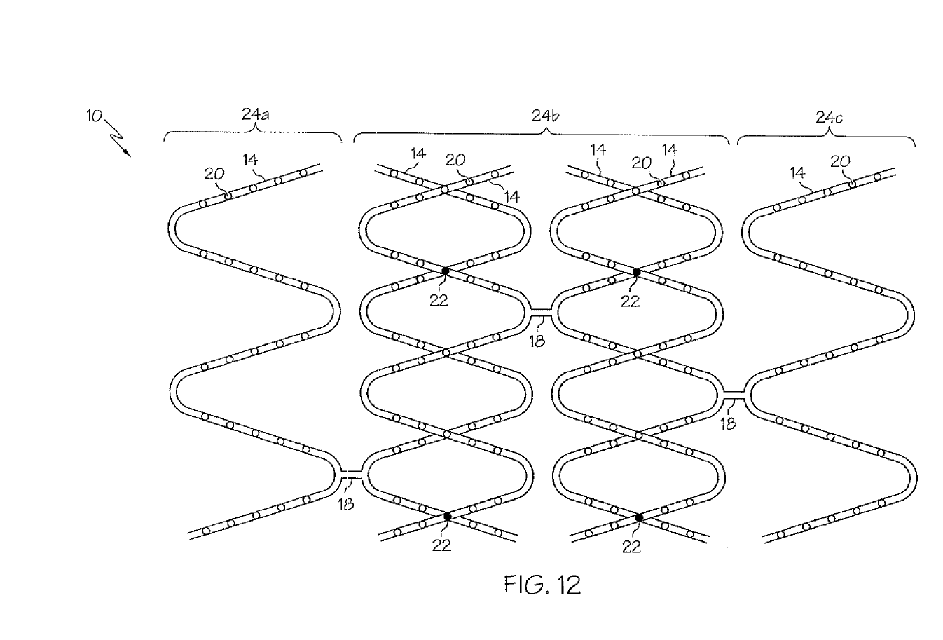

Note that the density of holes 20 when the stent 10 is in the expanded state affects the amount of therapeutic agent eluted per unit of area. Thus, a greater density of holes 20 elutes a greater amount of therapeutic agent than a lower density of holes 20. In at least one embodiment, a stent 10 is constructed and arranged so that a first portion of the stent 10 has a greater density of holes 20 when the stent 10 is in the expanded state as compared to a second portion of the stent 10. This is shown, for example, in FIGS. 11-12 where the middle section 24b has a greater density of holes 20 when the stent 10 is in the expanded state than the proximal and distal sections 24a,c. In one embodiment the section(s) of the stent 10 with a greater density of holes 20 when the stent 10 is in the expanded state is positioned against an area of the vessel wall that requires greater amount of therapeutic agent.

In at least one embodiment, the density of the holes 20 when the stent 10 is in the expanded state is affected by degree or amount of the overlap of adjacent members 12 when the stent 10 is in the unexpanded state In some embodiments, the members 12 of the stent 10 do not overlap when the stent 10 is in the expanded state, as shown for example, in FIG. 4. In other embodiments, the members 12 of the stent 10 overlap when the stent 10 is in the expanded state, as shown, for example, in FIG. 2. In this embodiment, the amount of overlap is greater in the unexpanded state than in the expanded state.

In some embodiments, the stent 10 has a first number of holes 20 when the stent 10 is in the unexpanded state and a second number of holes 20 when the stent 10 is in the expanded state where the second number is greater than the first number. This is shown, for example, in FIGS. 1 and 2 where the stent 10 has a first number of holes 20 when the stent 10 is in the unexpanded state, as shown in FIG. 1 and a second number of holes 20 when the stent 10 is in the expanded state, as shown in FIG. 2, where the first number is less than the second number. As shown, the first number of holes 20 is less than the second number of holes 20 because the holes 20 in overlapping members 12 are aligned and thereby forming a through hole through two members 12 when the stent 10 is in the unexpanded state. Thus, in this embodiment, the second number of holes 20 is twice the first number of holes 20. Note that the holes 20 in this embodiment are radially overlapping when the stent 10 is in the unexpanded state.

In other embodiments, the stent 10 has the same number of holes 20 when the stent 10 is in the unexpanded state and when the stent 10 is in the expanded state. This is shown, for example, in FIGS. 3 and 4 where the holes 20 are not aligned when the stent 10 is in the unexpanded state, shown in FIG. 3.

In at least one embodiment, the holes 20 have a first depth when the stent 10 is in the unexpanded state and a second depth when the stent is in the expanded state where the first depth is greater than the second depth, as shown, for example, in FIGS. 1 and 2. In this embodiment, the holes 20 have a first depth greater than the second depth because holes 20 in overlapping members 12 are aligned when the stent 10 is in the unexpanded state so that the holes 20 extend from the exterior surface of the first member 12a to the exterior surface of the second member 12b In other embodiments, the holes 20 of the first member 12a are offset from the holes 20 of the second member 12b when the stent 10 is in the unexpanded state, as shown for example, in FIG. 5. In this embodiment, the depth of the holes 20 when the stent 10 is in the unexpanded state is equal to the depth of the holes 20 when the stent 10 is in the expanded state.

As discussed above, members 12 can overlap in different ways. In some embodiments, two members 12 are entirely overlapped when the stent 10 is in the unexpanded state. This is shown, for example, in FIG. 1, where the length of the first strut 14a overlaps the length of the second strut 14b. In some embodiments, at least some of the holes 20 of the first strut 14a radially overlap at least some of the holes 20 of the second strut 14b when the stent 10 is in the unexpanded state and at least one of the holes 20 of the first strut 14a radially overlap at least one of the holes 20 of the second strut 14a when the stent 10 is in the expanded state This is shown, for example, by hole 20a in FIGS. 1 and 2. Hole 20a is formed by the holes 20 of the first and second struts 14a,b which are radially overlapping when the stent 10 is in the unexpanded state and when the stent 10 is in the expanded state. It is also within the scope of the invention at least some of the holes 20 of the first strut 14a radially overlap at least some of the holes 20 of the second strut 14b when the stent 10 is in the unexpanded state and for none of the holes 20 of the first and second struts 14a,b to be radially overlapping when the stent 10 is in the expanded state.

In other embodiments, only a portion of one member 12 overlaps only a portion of another member 12 when the stent 10 is in the unexpanded state. This is shown, for example in FIGS. 3 and 4 where only a portion of the first strut 14a overlaps only a portion of the adjacent strut 14b when the stent 10 is in the unexpanded state.

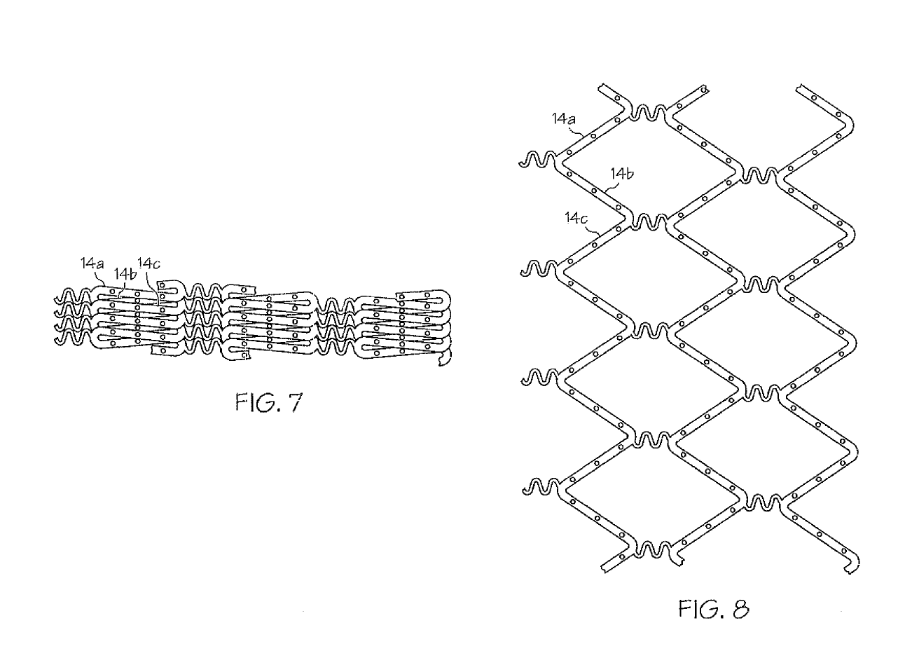

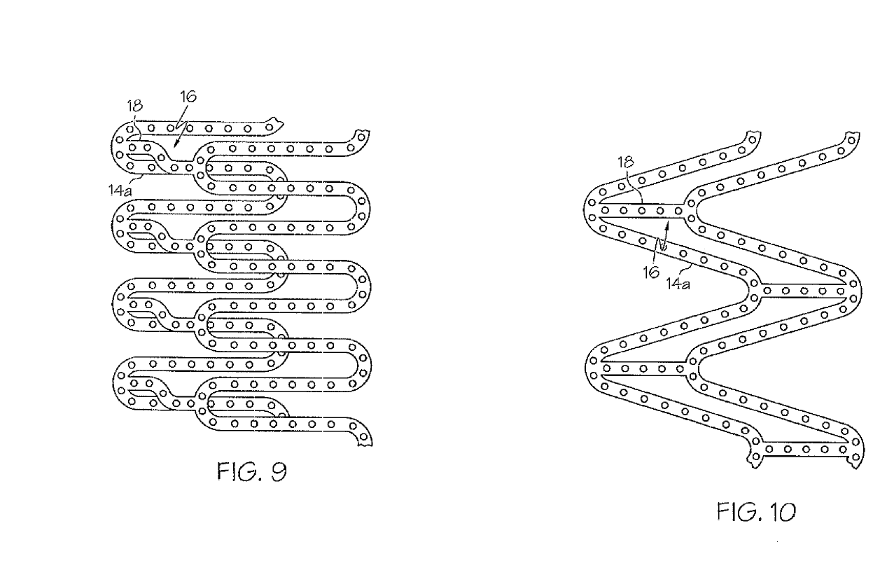

In still other embodiments, at least two portions of a member 12 are overlapped by adjacent members 12 when the stent 10 is in the unexpanded state. This is shown, for example, in FIG. 5 where the a first portion of the second strut 14b is overlapped by a portion of the adjacent first strut 14a and a second portion of the second strut 14b is overlapping a portion of the adjacent third strut 14c when the stent 10 is in the unexpanded state. In FIG. 7, a portion of the first strut 14a overlaps a first portion of the second strut 14b and a portion of the third strut 14c overlaps a second portion of the second strut 14b when the stent 10 is in the unexpanded state. In some embodiments, a portion of a connector 18 overlaps a portion of a strut 14 when the stent 10 is in the unexpanded state. This is shown, for example, in FIG. 9, where a portion of connector 18 overlaps a portion of strut 14 when the stent 10 is in an unexpanded state.

In some embodiments, at least a portion of the members 12 of the stent 10 are overlapping when the stent 10 is in the expanded state. This is shown, for example, in FIGS. 1-2 where a portion of the first and second struts 14a,b are overlapping when the stent 10 is in the expanded state In other embodiments, the members 12 of the stent 10 are non-overlapping when the stent 10 is in the expanded state. This is shown, for example, in FIGS. 6, 8 and 10, where the members 12 are no longer overlapping when the stent 10 is in an expanded state. In FIG. 10, the connector 18 straightens and the struts 14 expand so that the members 12 are no longer overlapping when the stent 10 expands.

The inventive stents 10 may be made from any suitable biocompatible materials including one or more polymers, one or, more metals or combinations of polymer(s) and metal(s). Examples of suitable materials include biodegradable materials that are also biocompatible By biodegradable is meant that a material will undergo breakdown or decomposition into harmless compounds as part of a normal biological process. Suitable biodegradable materials include polylactic acid, polyglycolic acid (PGA), collagen or other connective proteins or natural materials, polycaprolactone, hyaluronic acid, adhesive proteins, co-polymers of these materials as well as composites and combinations thereof and combinations of other biodegradable polymers Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

The inventive stents 10 may be made of shape memory materials such as superelastic Nitinol or spring steel, or may be made of materials which are plastically deformable. In the case of shape memory materials, the stent 10 may be provided with a memorized shape and then deformed to a reduced diameter shape. The stent 10 may restore itself to its memorized shape upon being heated to a transition temperature and having any restraints removed therefrom.

The inventive stents 10 may be created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the inventive stents disclosed herein.

In some embodiments the stent 10, the delivery system or other portion of the assembly may include one or, more areas, bands, coatings, members, etc. that is (are) detectable by imaging modalities such as X-Ray, MRI, ultrasound, etc. In some embodiments at least a portion of the stent 10 and/or adjacent assembly is at least partially radiopaque.

Often the therapeutic agent deposited into the holes 20 or onto surface(s) of the members 12 which are not overlapped, is in the form of a coating or other layer (or layers) of material placed on a surface region of the stent 10, which is adapted to be released at the site of the stent's implantation or areas adjacent thereto. A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where a therapeutic agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims) In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the invention. Those skilled in the art may recognize other equivalents to the specific embodiment described herein which equivalents are intended to be encompassed by the claims attached hereto

1. A stent, the stent having an unexpanded state and an expanded state, the stent comprising a plurality of struts, the plurality of struts comprising a first strut and a second strut, the first strut defining a plurality of first holes, the second strut defining a plurality of second holes, a plurality of the first and second holes being radially overlapped when the stent is in the unexpanded state, and at least one of the first and second of holes being radially overlapped when the stent is in the expanded state.

2. The stent of claim 1, only one of the first and second of holes being radially over lapped when the stent is in the expanded state.

3. The stent of claim 1, none of the first and second of holes being radially overlapped when the stent is in the expanded state.

4. The stent of claim 1, each of the plurality of first holes and each of the plurality of second holes having a therapeutic agent deposited therein.

5. The stent of claim 4, further comprising a plurality of turns, each turn engaging two of the plurality of struts, each having an outer surface that is not overlapped when the stent is in the unexpanded state, each turn having a therapeutic agent deposited on the outer surface.

6. A stent, the stent having an unexpanded state and an expanded state, the stent comprising a plurality of members, the plurality of members defining at least one hole, a therapeutic agent being deposited into the at least one hole, some of the plurality of members having first portions and second portions, the first portions being overlapping when the stent is in the unexpanded state and non-overlapping when the stent is in the expanded state, the second portions being non-overlapping when the stent is in the unexpanded state, the therapeutic agent being further deposited on the second portions of circumferentially adjacent members.
